(19) United States
(12) Patent Application Publication
(10) Pub. No.: US 2002/0151054 A1
(43) Pub. Date: Oct. 17, 2002

(54) Cellular production control

(76) Inventors: Peter David Rathjen, Blackwood (AU); Joy Rathjen, Blackwood (AU); Kathryn May Hudson, Murray Bridge (AU)

Correspondence Address: Lisa M. Cobern, SUTHERLAND ASBILL & BRENNAN LLP, 999 Peachtree Street, NE, Atlanta, GA, 30309-3996, US

(21) Appl. No.: 10/090,849

(22) Filed: Mar. 4, 2002

(30) Foreign Application Priority Data
Mar. 2, 2001 (AU) PR3495

(51) Int. Cl. C12N 5/08

(52) U.S. Cl. 435/366; 435/325; 435/368

(57) ABSTRACT

A method for the preparation of cells of ectodermal or endodermal lineages, of high purity which method includes

providing

a source of pluripotent cells;

a source of a mesodermal suppression composition including cellular fibronectin (cFN); and

a suitable culture medium; and

culturing the pluripotent cells in the culture medium in the presence of the mesodermal suppression composition for a time sufficient to permit differentiation to ectoderm cells.

### CROSS REFERENCE TO RELATED APPLICATIONS

This application claims the priority benefit of Australian Patent Application PR3495 filed Mar. 2, 2001.

### BACKGROUND OF THE INVENTION

1. Field of the Invention

The present invention relates to a mesodermal suppression composition that is able to programme pluripotent cell differentiation to an ectoderm or endoderm cell, to a method of preparation of differentiated or partially differentiated ectoderm or endoderm cells, and to the differentiated or partially differentiated ectoderm or endoderm cells derived therefrom. The present invention also relates to uses of the mesodermal suppression composition, to methods of producing and culturing the ectoderm or endoderm cells of the invention, and to uses thereof.

2. Background Art

Initial developmental events within the mammalian embryo entail the elaboration of extra-embryonic cell lineages and result in the formation of the blastocyst, which comprises trophectoderm, primitive endoderm and a pool of pluripotent cells, the inner cell mass (ICM/epiblast). As development continues, the cells of the ICM/epiblast undergo rapid proliferation, selective apoptosis, differentiation and reorganisation as they develop to form the primitive ectoderm. In the mouse, the cells of the ICM begin to proliferate rapidly around the time of blastocyst implantation. The resulting pluripotent cell mass expands into the blastocoelic cavity. Between 5.0 and 5.5 days post coitum (dpc) the inner cells of the epiblast undergo apoptosis to form the proamniotic cavity. The outer, surviving cells, or early primitive ectoderm, continue to proliferate and by 6.0-6.5 dpc have formed a pseudo-stratified epithelial layer of pluripotent cells, termed the primitive or embryonic ectoderm. Primitive ectoderm cells are pluripotent, and distinct from cells of the ICM, giving rise to the germ cells and acting as a substrate for the generation of the primary germ layers of the embryo proper (mesoderm, endoderm and ectoderm) and the extra-embryonic mesoderm during gastrulation.

By 4.5 dpc pluripotent cells exposed to the blastocoelic cavity have differentiated to form primitive endoderm. The primitive endoderm gives rise to two distinct endodermal cell populations, visceral endoderm, which remains in contact with the epiblast, and parietal endoderm, which migrates away from the pluripotent cells to form a layer of endoderm adjacent to the trophectoderm. Formation of these endodermal layers is coincident with the formation of primitive ectoderm and the creation of an inner cavity.

In the human and in other mammals, formation of the blastocyst, including development of ICM cells and their progression to pluripotent cells of the primitive ectoderm and subsequent differentiation to form the embryonic germ layers, follow a similar developmental process.

Pluripotent cells can be isolated from the preimplantation mouse embryo as embryonic stem (ES) cells. ES cells can be maintained indefinitely as a pluripotent cell population in vitro, and, when reintroduced into a host blastocyst, can contribute to all adult tissues of the mouse including the germ cells. ES cells, therefore, retain the ability to respond to all the signals that regulate normal mouse development, and potentially represent a powerful model system for the investigation of mechanisms underlying pluripotent cell biology and differentiation within the early embryo, as well as providing opportunities for embryo manipulation and resultant commercial, medical and agricultural applications. ES cells and other pluripotent cells and cell lines will share some or all of these properties and applications.

To date, the successful isolation, long term clonal maintenance, genetic manipulation and germ-line transmission of pluripotent cells from species other than rodents has generally been difficult and the reasons for this are unknown. International patent application WO97/32033 and U.S. Pat. No. 5,453,357 describe pluripotent cells including cells from species other than rodents, and primate pluripotent cells have been described in International patent applications WO98/43679 and WO96/23362 and in U.S. Pat. No. 5,843,780.

The differentiation of ES cells can be regulated in vitro by the cytokine leukaemia inhibitory factor (LIF) and other gp130 agonists which promote self-renewal and prevent differentiation of the stem cells. However, there is little information about biological molecules that can induce the differentiation of ES cells into specific cell types.

Applicants have previously demonstrated, in contrast to ES cells, that early primitive ectoderm-like (EPL) cells will preferentially form mesoderm when differentiated as embryoid bodies. See International patent application PCT/AU99/00265, the entire disclosure of which is incorporated herein by reference. This has been demonstrated morphologically with the formation of beating cardiocytes (mesoderm) but not neurons (ectoderm), and by gene expression analysis, with the expression of brachyury, an early mesoderm specific marker but not Sox1, an early neurectoderm marker. Furthermore, applicants have shown that the mesodermal progenitor cells formed during EPL cell differentiation can be directed to form alternate mesodermal cell lineages, such as macrophages, by the addition of exogenous signalling molecules.

Chemical inducers such as retinoic acid have been used to form limited neural lineages. However, the variety of neural cell types produced by these methods is limited.

### DETAILED DESCRIPTION OF THE INVENTION

It is an object of the present invention to overcome, or at least alleviate, one or more of the difficulties or deficiencies associated with the prior art.

Applicant has surprisingly identified a mesodermal suppression factor derived from a conditioned medium, as hereinafter described, which may be used to modulate differentiation of EPL cells to mesodermal lineages and/or permit differentiation of EPL cells to ectodermal or endodermal lineages.

In a first aspect of the present invention, there is provided a method for the preparation of cells of ectodermal or endodermal lineages, of high purity which method includes

providing

a source of pluripotent cells;

a source of a mesodermal suppression composition including cellular fibronectin (cFN); and

a suitable culture medium; and

culturing the pluripotent cells in the culture medium in the presence of the mesodermal suppression composition for a time sufficient to permit differentiation to ectoderm cells.

Whilst we do not wish to be restricted by theory, it is postulated that the mesodermal suppression composition permits the preferential preparation of ectoderm or endoderm cells by suppression of mesoderm formation. This results in e.g. an ectoderm cell population of high purity. As used herein, the term “high purity” refers to a cell population that does not contain at least some of the constituents with which it is found in nature.

The cells of ectodermal lineage may be neurectoderm or surface ectoderm or their partially or terminally differentiated progeny. The cells of endodermal lineages may be primitive endoderm, visceral endoderm or parietal endoderm or their partially or terminally differentiated progeny.

According to this aspect of the present invention, applicants have demonstrated that the lack of ectodermal lineages in EPL cell differentiation results from deficiencies in the embryoid body environment and not from an inherent restriction in the differentiation potential of EPL cells. Comparison of EPL cell embryoid bodies with ES cell embryoid bodies, which form both mesodermal and ectodermal lineages, showed EPL cell embryoid bodies lack visceral endoderm. Visceral endoderm is a known source of instructive signalling molecules involved in cell fate specification during early embryogenesis. Visceral endoderm-like signals, derived from MEDII conditioned medium, were analysed for components capable of modulating mesoderm induction from EPL cells.

It was surprisingly discovered that cFN may function as a mesoderm suppression agent. For example, applicants found that aggregation of EPL cells in a medium containing MEDII or R significantly reduced the number of embryoid bodies giving rise to macrophages when compared to aggregates formed in medium without supplement or supplemented with E. The reduction in macrophage formation was also observed in embryoid bodies aggregated in cFN, but not in MEDII or R depleted in cFN. This demonstrated a role for cFN in the regulation of mesoderm formation from EPL cells. Formation of ES cell embryoid bodies in equivalent medium failed to demonstrate a role for cFN in the regulation of mesoderm. The presence of visceral endoderm in these aggregates is predicted to be producing endogenous signalling molecules that independently direct pluripotent cell differentiation and result in the generation of multiple lineages.

The pluripotent cells from which the ectoderm or endoderm cells may be derived may be selected from the group consisting of in vivo or in vitro derived ICM/epiblast, in vivo or in vitro derived primitive ectoderm, primordial germ cells, EG cells, teratocarcinoma cells, EC cells, and pluripotent cells derived by dedifferentiation or by nuclear transfer. Early EPL cells are also pluripotent stem cells and are preferred. They differ in some properties to ES cells, and have the capacity to revert to ES cells in vitro. They can be derived from ES cells or other types of pluripotent cells, and are the in vitro equivalent of primitive ectoderm cells of post-implantation embryos. As such, EPL cells can also be established in vitro from cells isolated from the primitive ectoderm of post-implantation embryos. The properties of EPL cells, factors required for their maintenance and proliferation in vitro, and their ability to differentiate uniformly in vitro to form essentially homogeneous populations of partially differentiated and differentiated cell types are described fully in PCT/AU99/00265 above. Cells of the primordial gonad and primordial germ cells (PGCs), also retain pluripotency during embryonic development, and can be isolated and cultured in vitro as embryonic gonadal (EG) cells. Embryonic carcinoma (EC) cells may also be pluripotent.

The pluripotent cell source may take the form of embryoid bodies (EBs) derived from ES or EPL cells in vitro, or cellular aggregation.

The pluripotent cells may be from any vertebrate including murine, human, bovine, ovine, porcine, caprine, equine and chicken. The cells may be isolated by any method known to the skilled addressee.

The source of cFN may be cFN itself, or a conditioned medium as described in International patent application PCT/AU99/00265 above, or an extract thereof containing cFN.

Preferably the conditioned medium is prepared using an hepatic or hepatoma cell or cell line, more preferably a human hepatocellular carcinoma cell line such as Hep G2 cells (ATCC HB-8065) or Hepa-1c1c-7 cells (ATCC CRL2026), primary embryonic mouse liver cells, primary adult mouse liver cells, or primary chicken liver cells, or an extraembryonic endodermal cell or cell line such as the cell lines END-2 and PYS-2.

The pluripotent, e.g. EPL cells may be cultured according to the present invention under conditions suitable for their proliferation and maintenance in vitro. This includes the use of serum including fetal calf serum and bovine serum or the medium may be serum-free. Other growth enhancing components such as insulin, transferrin and sodium selenite may be added to improve growth of the cell types preferred. As would be readily apparent to a person skilled in the art, the growth enhancing components will be dependent upon the cell types cultured, other growth factors present, attachment factors and amounts of serum present.

The cells may be cultured for a time sufficient to establish the cells in culture. By this, we mean a time when the cells equilibrate in the culture medium. Preferably, the cells are cultured for approximately 2-6 days.

The cell culture medium may be any cell culture medium appropriate to sustain the cells employed. Where the cells are a liver cell or liver cell line, the culture medium is preferably DMEM containing high glucose, 40 μg/ml gentamycin, 1 mM L-glutamine, 37° C., 5% CO₂. The medium may contain up to 10% Foetal Calf Serum (FCS), but preferably the medium is serum free.

Separation of the cell culture medium from the cells may be achieved by any suitable technique, such as decanting the medium from the cells. Preferably the cell culture medium is clarified by centrifugation or filtration (e.g. through a 0.22 μM filter) to remove excess cells and cellular debris. Other known means of separating the cells from the medium may be employed providing the separation method does not remove the growth components from the medium.

In a preferred embodiment of this aspect of the present invention, the method may further include

providing a growth factor; and

culturing the pluripotent cells in the culture medium in the presence of the cFN source and growth factor.

The growth factor may be selected to direct a specific ectodermal or endodermal fate. Where neurectoderm formation is required, a growth factor selected from the FGF family may be used.

The growth factor from the FGF family may be of any suitable type. Examples include aFGF, bFGF and FGF4. FGF4 and/or bFGF is particularly preferred.

The concentration of the growth factor from the FGF family is preferably in the range approximately 1 to 100 ng/ml, more preferably approximately 5 to 50 ng/ml. When bFGF is used, its concentration is preferably approximately 10 ng/ml.

As used herein, the term “neurectoderm” refers to undifferentiated neural progenitor cells substantially equivalent to cell populations comprising the neural plate and/or neural tube. They are multipotential, and have the capacity to differentiate into all cell types that make up the central nervous system and peripheral nervous system, including neuronal cells, glial cells and neural crest cells (as outlined in Australian Provisional Application PQ7143 “Cell Production” to applicants, the entire disclosure of which is incorporated herein by reference).

In a further aspect of the present invention there are provided methods for proliferating cells of ectodermal and/or endodermal lineage and/or producing differentiated or partially differentiated cells from ectodermal or endodermal cells.

Methods for proliferating neurectoderm cells, for example, are described in Australian provisional application PQ7143, the entire disclosure of which is incorporated herein by reference.

The differentiated or partially differentiated cells so formed may be neuronal cells, neural crest cells, glial cell precursors, cells of glial lineage or differentiated neurons or glial cells, visceral or parietal endoderm.

In a further aspect of the present invention there is provided a neurectoderm cell, a partially differentiated neurectoderm cell, a terminally differentiated neuronal cell, a partially differentiated neural crest cell, or a terminally differentiated neural crest cell, a partially differentiated glial cell, or a terminally differentiated glial cell, a visceral endoderm or parietal endoderm produced by the methods of the present invention.

The neurectoderm cell or differentiated or partially differentiated cell derived therefrom may be from any vertebrate including murine, human, bovine, ovine, porcine, caprine, equine and chicken.

The neurectoderm cells of the present invention and the differentiated or partially differentiated cells derived therefrom, have a number of uses, including the following:

use as cytoplasts or karyoplasts in nuclear transfer

use as a source of nuclear material for nuclear transfer

use to produce cells, tissues or components of organs for transplant

use in human cell therapy to treat neuronal diseases. For example neuronal cells may be used in cell therapy to treat Parkinson's disease

use in human gene therapy to treat neuronal diseases

The present invention will now be more fully described with reference to the accompanying examples and drawings. It should be understood, however, that the description following is illustrative only and should not be taken in any way as a restriction on the generality of the invention described above.

### IN THE FIGURES

FIG. 1 is a graph showing the percentage of macrophage forming bursts achieved from EPL cells under various growth conditions.

FIGS. 2a to g are electron micrographs showing the level of brachyury expression under similar growth conditions.

### DEFINITIONS

MII MEDII, conditioned medium from HepG2 cells

cFN Cellular fibronectin, a biologically active component in MEDII

R Retentate, after filtration through a 10 kDa filter, the large molecular weight fraction of serum-free MEDII. This fraction also contains cFN.

E Eluate, after filtration through a 10 kDa filter, the small molecular weight fraction of serum-free MEDII

Therefore E+R=MEDII

R-cFN Retentate stripped of cFN

MII-cFN Serum free MEDII stripped of cFN

### EXAMPLE 1

### Terminal Differentiation of Mesodermal Lineages—Macrophage Formation

As described in Lake et al (2000), mesoderm formation is upregulated when EPL cells are cultured and allowed to form embryoid bodies in incomplete medium. Ectodermal lineages (as measured by neuron formation) were not detected in these aggregates.

Aim

To examine the effect of the addition of MEDII and its components on the level of mesoderm formation as determined by the level of macrophage burst formation, a terminally differentiated mesoderm cell type.

Method

EPL cells were cultured in MEDII for 3 days on gelatinised plates, a single cell suspension was made from these cells. Aggregates were then allowed to form in incomplete medium [Gibco's Dulbecco's Modified Eagle Medium (D-MEM) liquid (high glucose) without HEPES buffer, supplemented with 10% foetal calf serum (FCS), 0.1 mM β-mercaptoethanol (β-ME) and 1 mM L-glutamine], with the addition of MEDII factors to the medium, Retentate (used at 50 μg/ml), Eluate (used at 50%), MEDII (50%, cFN (10 μg/ml), Retentate-cFN (50 μg/ml), MEDII-cFN (50%).

After 48 hours in this medium, approximately 50 aggregates were transferred into 1.25 ml MC media [0.9% methyl cellulose in Iscove's modified DMEM (IMDM), 15% FCS, 50 μg/ml ascorbic acid, 4.5×10⁻⁴ M α-monothioglycerol] supplemented with 400 U/ml recombinant mouse IL-3 (courtesy of Dr T Gonda, Institute of Medical and Veterinary Science, Adelaide) and 10 ng/ml recombinant human M-CSF (R&D Systems), Colonies were scored for the presence or absence of macrophage bursts by microscopic examination on days 8, 10 and 12.

Results

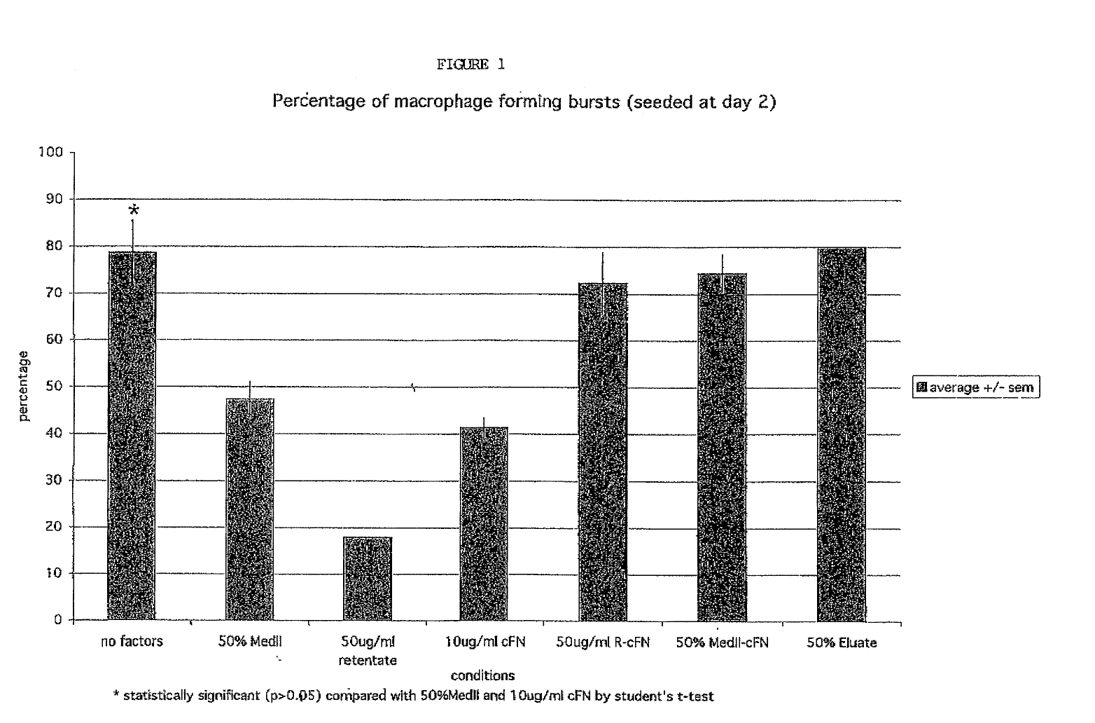

As expected aggregates derived from EPL cells and cultured in incomplete medium (EPLEB) formed mesoderm at a high level as determined by macrophage formation (on average 77%), however, the aggregates grown in the presence of MEDII, or cFN were significantly lower statistically (p<0.05) than those grown without factors and R-cFN. This was demonstrated by an unpaired, one-tailed students t-test. (Statistical analysis was not possible on other data points). The graph in FIG. 1 clearly demonstrates that aggregates grown in the presence of cFN (i.e. R, MII, cFN) have a suppression of mesodermal formation. This pattern was maintained if aggregates were culture in the factors for 3 or 4 days.

Macrophage activity was confirmed by phagocytosis assays as follows: opsonisation of zymosan A (Sigma Chemical Co. St Louis, Mo.) was carried out as previously described (Strzelecka et al 1997). Briefly, particles were incubated for 1 hour with 20 mg/ml human IgG (Sigma Chemical Co., St. Louis, Mo.) at a final concentration of 10 mg/ml zymosan A. Particles were then washed 3 times with PBS to remove unbound IgG and resuspended in PBS. Macrophages grown as described above, were plated on glass coverslips and allowed to bind opsonised zymosan A in PBS, for 20 minutes on ice. To initiate particle uptake, cells were transferred to pre-warmed DMEM/10% FCS and at the required time points, cells were processed for immunofluorescence analysis.

### EXAMPLE 2

### Identification of Early Mesoderm-Brachyury In Situ Hybridisation

Aim

The in situ probe for brachyury was used as an early transient marker for mesoderm formation.

Method

The aggregates were grown as described above, and collected at day 2, day 3 and day 4 after initial aggregate formation, i.e. while culturing in incomplete medium and the presence of factors. Immunohistochemistry was performed on free floating embryoid bodies. The bodies were fixed in 4% paraformaldehyde for 20 minutes and dehydrated into 70% ethanol. After rehydration the bodies were permeabilised using 0.1% active DEPC (Sigma Chemical Co., St Louis, Mo.) in PBS for 15 minutes. Hybridization was at 50° C. overnight using the DIG (Roche) labelled antisense transcript from the BamHI cut psk75 brachyury plasmid in 50% formamide. Bodies were washed and DIG detected using anti-DIG-AP antibody (Roche) following the manufacturers recommendations.

Results

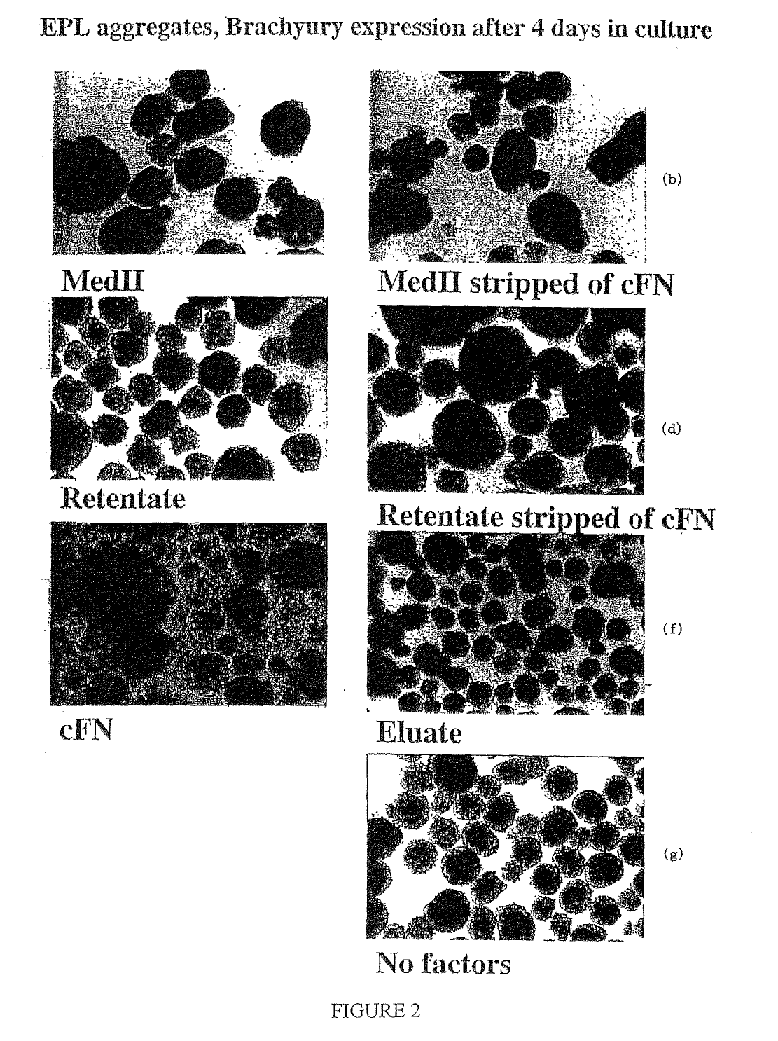

The level of brachyury expression, indicative of early mesoderm formation is comparable to the level of macrophage forming bursts. For example, the conditions which did not have the addition of cFN, had high levels of brachyury expression. Those which had cFN in the medium had low levels of brachyury expression, i.e. no factors (EPLEB), R-cFN, SF-cFN and Eluate. These results are shown in FIG. 2a to g.

Conclusion

As shown by both brachyury expression (early mesoderm formation), and by macrophage forming bursts (terminal differentiation of mesoderm), the addition of cFN or parts of MEDII which contain cFN to EPL cell aggregates at very early stages of development significantly reduce the level of mesoderm formation.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

It will be understood that the invention disclosed and defined herein extends to all alternative combinations of two or more of the individual features mentioned or evident from the text or drawings. All of these different combinations constitute various alternative aspects of the invention.

### REFERENCES

Lake, J. A., Rathjen, J., Remiszewski, J. and Rathjen, P. D. (2000) Reversible programming of pluripotent cell differentiation. J. Cell Science 113, 555-566.

Strzelecka et al., (1997) Syk kinase, tyrosine-phosphorylated proteins and actin filaments accumulate at forming phagosomes during Fcgamma receptor-mediated phagocytosis. Cell Motil Cytoskeleton 38, 287-96.

1. A method for the preparation of cells of ectodermal or endodermal lineages, of high purity which method includes
providing
a source of pluripotent cells;
a source of a mesodermal suppression composition including cellular fibronectin (cFN); and
a suitable culture medium; and
culturing the pluripotent cells in the culture medium in the presence of the mesodermal suppression composition for a time sufficient to permit differentiation to ectoderm cells.

2. A method according to claim 1, wherein the cells of ectoderm lineage are selected from neurectoderm or surface ectoderm, or their partially or terminally differentiated progeny.

3. A method according to claim 1, wherein the cells of endoderm lineages are selected from primitive endoderm, visceral endoderm or parietal endoderm, or their partially or terminally differentiated progeny.

4. A method according to claim 1, wherein the source of pluripotent cells is selected from the group consisting of in vivo or in vitro derived ICM/epiblast, in vivo or in vitro derived primitive ectoderm, primordial germ cells, embryonic gonadal (EG) cells, teratocarcinoma cells, embryonic carcinoma (EC) cells, early primitive ectoderm-like (EPL) cells, and pluripotent cells derived by dedifferentiation or by nuclear transfer.

5. A method according to claim 4, wherein the source of pluripotent cells is early primitive ectoderm-like (EPL) cells.

6. A method according to claim 1, wherein the mesodermal suppression composition source is selected from the group consisting of cellular fibronectin (cFN), MEDII, a conditioned medium, or an extract therefrom containing cellular fibronectin (cFN).

7. A method according to claim 1, wherein the pluripotent cells are cultured in the culture medium for approximately 2 to 6 days.

8. A method according to claim 1 where in the culture medium is DMEM containing a high glucose content.

9. A method for the preparation of cells of ectodermal or endodermal lineages, of high purity which method includes
providing
a source of pluripotent cells;
a source of mesodermal suppression composition including cellular fibronectin (CFN);
a suitable culture medium; and
a growth factor; and
culturing the pluripotent cells in the culture medium in the presence of the cFN source and growth factor.

10. A method according to claim 9 wherein the cell produced is a neurectoderm cell.

11. A method according to claim 10, wherein the growth factor is from the FGF family.

12. A method according to claim 11, wherein the growth factor is selected from aFGF, bFGF and FGF4.

13. A method according to claim 9, wherein the growth factor is selected from bFGF and FGF4.

14. A method according to claim 13, wherein the growth factor is bFGF.

15. A method according to claim 11, wherein the FGF growth factor is present in a concentration in the range of approximately 1 to 100 ng/ml.

16. A method according to claim 15, wherein the concentration of the growth factor is in the range of 5 to 50 ng/ml.

17. A method according to claim 14, wherein concentration of bFGF is approximately 10 ng/ml.

18. A cell of ectodermal or endodermal lineages produced by a method according to claim 1.

19. A cell according to claim 18 wherein the cell is selected from the group consisting of a neurectoderm cell, a partially differentiated neurectoderm cell, a terminally differentiated neuronal cell, a partially differentiated neural crest cell, a terminally differentiated neural crest cell, a partially differentiated glial cell, or a terminally differentiated glial cell, a visceral endoderm cell, or a parietal endoderm cell.

20. A cell according to claim 19 wherein the cell is a neurectoderm cell.

21. Use of a neurectoderm cell according to claim 20 in nuclear transfer.

22. Use of a neurectoderm cell according to claim 20 in the production of cells, tissues or components of organs for transplant.

23. Use of a neurectoderm cell according to claim 20 in human cell therapy to treat neuronal diseases.

24. Use of a neurectoderm cell according to claim 20 in human gene therapy to treat neuronal diseases.

25. A method for the treatment of neuronal diseases, including Parkinson's disease, which method includes treating a patient requiring such treatment with neurectoderm cells according to claim 20, or their partially of terminally differentiated progeny, through human, or animal cell or gene therapy.
